(19) United States
(12) Patent Application Publication
(10) Pub. No.: US 2026/0276584 A1
(43) Pub. Date: Sep. 17, 2026

(54) PAPER BASED MICROCHIP ELECTROPHORESIS FOR MOLECULAR DIAGNOSTIC TESTS

(71) Applicant: UNIVERSITY OF HOUSTON SYSTEM, Houston, TX (US)

(72) Inventors: Ran An, Houston, TX (US); Yi Yang, Houston, TX (US); Richard Willson, Houston, TX (US)

(73) Assignee: UNIVERSITY OF HOUSTON SYSTEM, Houston, TX (US)

(21) Appl. No.: 19/472,235

(22) PCT Filed: Apr. 2, 2024
(86) PCT No.: PCT/US2024/022675

§ 371 Date: Oct. 3, 2025

Related U.S. Application Data
(60) Provisional application No. 63/456,734, filed on Apr. 3, 2023.

(51) Int. Cl. G01N 27/447; C12Q 1/6806; C12Q 1/6851

(52) U.S. Cl. CPC G01N 27/44747; C12Q 1/6806; C12Q 1/6851; G01N 27/44721; G01N 27/44791

(57) ABSTRACT

Embodiments of the present disclosure pertain to methods of separating nucleic acids from a sample by applying the sample to a fiber-based electrophoresis medium and generating an electric field across the fiber-based electrophoresis medium, where the generated electric field induces migration and separation of the nucleic acids across the fiber-based electrophoresis medium. The methods of the present disclosure may also include a step of detecting one or more of the separated nucleic acids on the fiber-based electrophoresis medium, diagnosing a disease or condition based on the detecting, and implementing a treatment decision based on the diagnosis. Additional embodiments of the present disclosure pertain to nucleic acid separation systems for separating nucleic acids and methods of making them.

### CROSS-REFERENCE TO RELATED APPLICATIONS

This application claims priority to U.S. Provisional Patent Application No. 63/456,734, filed on Apr. 3, 2023. The entirety of the aforementioned application is incorporated herein by reference.

### BACKGROUND

Nucleic acid gel electrophoresis has limitations in terms of costs, portability, detection speed, and detection specificity. Numerous embodiments of the present disclosure aim to address such limitations.

### SUMMARY

In some embodiments, the present disclosure pertains to methods of separating nucleic acids from a sample. In some embodiments, the methods of the present disclosure include applying the sample to a fiber-based electrophoresis medium and generating an electric field across the fiber-based electrophoresis medium, where the generated electric field induces migration and separation of the nucleic acids across the fiber-based electrophoresis medium. In some embodiments, the methods of the present disclosure also include a step of detecting one or more of the separated nucleic acids on the fiber-based electrophoresis medium. In some embodiments, the methods of the present disclosure also include a step of diagnosing a disease or condition based on the detecting. In some embodiments, the methods of the present disclosure also include a step of implementing a treatment decision based on the diagnosis. In some embodiments, the methods of the present disclosure also include a step of repeating the nucleic acid separation after implementing the treatment decision.

Additional embodiments of the present disclosure pertain to nucleic acid separation systems for separating nucleic acids. In some embodiments, the nucleic acid separation systems of the present disclosure include a fiber-based electrophoresis medium that is operational to receive a sample containing nucleic acids. In some embodiments, the nucleic acid separation systems of the present disclosure also include a first electrode and a second electrode operational to generate an electric field across the fiber-based electrophoresis medium. In some embodiments, the generated electric field induces migration and separation of the nucleic acids across the fiber-based electrophoresis medium. Additional embodiments of the present disclosure pertain to methods of making the nucleic acid separation systems of the present disclosure.

### DRAWINGS

FIG. 1 illustrates a method of separating nucleic acids from a sample in accordance with various embodiments of the present disclosure.

FIG. 2 illustrates a nucleic acid separation system in accordance with various embodiments of the present disclosure.

FIG. 3 shows an image of nucleic acids separated by a nucleic acid separation system of the present disclosure.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components that include one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Nucleic acid gel electrophoresis is a widely used technique in molecular biology research, which allows for the detection and analysis of DNA and RNA molecules in a variety of contexts, such as gene expression analysis, PCR product analysis, and DNA sequencing. However, nucleic acid gel electrophoresis has limitations in terms of costs, portability, detection speed, and detection specificity. Numerous embodiments of the present disclosure aim to address such limitations.

### Metods of Separating Nucleic aAids from a Sample

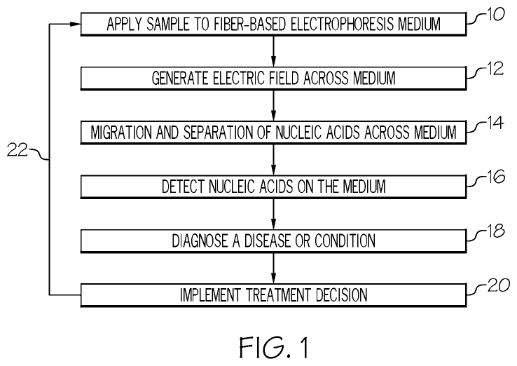

In some embodiments, the present disclosure pertains to methods of separating nucleic acids from a sample. In some embodiments illustrated in FIG. 1, the methods of the present disclosure include: applying the sample to a fiber-based electrophoresis medium (step 10); and generating an electric field across the fiber-based electrophoresis medium (step 12), where the generated electric field induces migration and separation of the nucleic acids across the fiber-based electrophoresis medium (step 14). In some embodiments, the methods of the present disclosure also include a step of detecting one or more of the separated nucleic acids on the fiber-based electrophoresis medium (step 16). In some embodiments, the methods of the present disclosure also include a step of diagnosing a disease or condition based on the detecting (step 18). In some embodiments, the methods of the present disclosure also include a step of implementing a treatment decision based on the diagnosis (step 20). In some embodiments, the methods of the present disclosure also include a step of repeating the nucleic acid separation after implementing the treatment decision (step 22). As set forth in more detail herein, the methods of the present disclosure can have numerous embodiments.

### Generation of an Electric Field

Various methods may be utilized to generate an electric field across a fiber-based electrophoresis medium. For instance, in some embodiments, a first and a second electrode are utilized to generate the electric field across the fiber-based electrophoresis medium. In some embodiments, the first and second electrode are placed on opposite ends of the fiber-based electrophoresis medium. In some embodiments, the first electrode is an anode and the second electrode is a cathode. In some embodiment, the nucleic acids in a sample migrate away from the anode and towards the cathode.

### Fiber-Based Electrophoresis Medium

The methods of the present disclosure may utilize various fiber-based electrophoresis media. For instance, in some embodiments, the fiber-based electrophoresis medium includes a paper-based electrophoresis medium. In some embodiments, the fiber-based electrophoresis medium includes, without limitation, cellulose acetate, cellulose, nitrocellulose, polyvinylidene difluoride (PVDF), nylon, or combinations thereof. In some embodiments, the fiber-based electrophoresis medium includes cellulose acetate. In some embodiments, the fiber-based electrophoresis medium is in the form of a microchip. In some embodiments, the fiber-based electrophoresis medium serves as a sieving medium in order to provide sieving capability for electrophoretic separation.

some embodiments, the fiber-based electrophoresis medium is soaked with a buffer. In some embodiments, both ends of the electrophoresis medium are immersed in containers that include a buffer. In some embodiments, the buffer includes, without limitation, tris, borate, Ethylenediaminetetraacetic acid (EDTA), or combinations thereof. In some embodiments, the buffer solution can include a tris/borate/EDTA buffer solution at a pH of 8.4.

### Samples

The methods of the present disclosure may be utilized to separate nucleic acids from various samples. For instance, in some embodiments, the sample includes a biological sample. In some embodiments, the biological sample is derived from a subject. In some embodiments, the biological sample includes tissues, cells, blood, or combinations thereof.

In some embodiments, the sample includes an environmental sample. In some embodiments, the environmental sample is obtained from a water source, a soil, or combinations thereof.

### Pre-Treatment of Samples

In some embodiments, samples may be pre-treated prior to the separation of nucleic acids. As such, in some embodiments, the methods of the present disclosure also include a step of pre-treating the sample. In some embodiments, the sample pre-treatment step includes purifying the nucleic acids from the sample, amplifying the nucleic acids, labeling the nucleic acids (e.g., incorporating labels or tags into nucleic acids or binding them to nucleic acids, optionally during amplification), mixing the nucleic acids with one or more detection dyes, mixing the nucleic acids with loading dyes, or combinations thereof.

In some embodiments, the sample pre-treatment step includes labeling the nucleic acids. In some embodiments, the nucleic acid labels may include, without limitation, fluors, stains, dycs, intercalators, particles, aptamers, antibodies, enzymes, catalytic nucleic acids, virions, or combinations thereof.

In some embodiments, the sample pre-treatment step includes purifying the nucleic acids from the sample. In some embodiments, the purification step includes a nucleic acid extraction step, an adsorption step, a lysis step, a filtration step, or a pathogen inactivation step.

In some embodiments, the pre-treatment step includes a step of amplifying the nucleic acids. Nucleic acids may be amplified in various manners. For instance, in some embodiments, nucleic acid amplification occurs by a method that includes, without limitation, isothermal amplification, loop-mediated isothermal amplification (LAMP), recombinase polymerase amplification (RPA), helicase-dependent amplification (HDA), rolling circle amplification (RCA), whole-genome amplification (WGA), multiple displacement amplification (MDA), nucleic acid sequence based amplification (NASBA), linear amplification, reverse transcription-based amplification, competitive amplification, polymerase chain reaction (PCR), competitive PCR, reverse transcription PCR (RT-PCR), asymmetric PCR, or combinations thereof.

The nucleic acid amplification steps of the present disclosure may utilize various catalysts. For instance, in some embodiments, the catalyst includes, without limitation, a polymerase with endonuclease activity, an endonuclease, an aptamer, a deoxyribozyme (i.e., DNAzyme), a ribozyme, or combinations thereof.

In some embodiments, the nucleic acids are amplified by a competitive amplification process. In some embodiments, the competitive amplification process utilizes the same primers for amplifying a target nucleic acid and another internal-standard sequence containing sequences with complementarity to the target-specific primers, which is added (preferably at a known concentration) to the amplification reaction, and usually amplifies with efficiency similar to that of the target nucleic acid. In some embodiments, the nucleic acid separation steps of the present disclosure separate nucleic acids amplified from the target from the nucleic acids amplified from the internal-standard sequence.

Competitive amplification processes may occur in various manners. For instance, in some embodiments, the competitive amplification process includes competitive PCR. In some embodiments, the competitive amplification process includes competitive isothermal amplification. In some embodiments, the competitive amplification process includes competitive reverse transcription. In some embodiments, the competitive amplification process includes competitive RPA.

In some embodiments, the sample pre-treatment step includes purifying the nucleic acids from the sample and then amplifying the purified nucleic acids. In some embodiments, the purified and amplified nucleic acids may then be loaded onto a fiber-based electrophoresis medium of the present disclosure.

The amplification processes of the present disclosure may utilize various primers. For instance, in some embodiments, the primers include modified primers. In some embodiments, the primers are modified with a tag, such as biotin, a fluorescent tag, a hapten that can be bound by an antibody, or combinations thereof. In some embodiments, the primers of the present disclosure include optimal amplicon sizes.

In some embodiments, the amplification reactions of the present disclosure include one or more hydrolysis probes. In some embodiments, the hydrolysis probes are designed to increase the specificity of an amplification step. In some embodiments, the hydrolysis probes include TaqMan probes. In some embodiments, the products of the hydrolysis of the hydrolysis probes are detected by electrophoresis.

Nucleic acids may be amplified under various conditions. For instance, in some embodiments, the amplification reactions include one or more surfactants suitable for facilitating purification, pathogen inactivation, amplification or separation. In some embodiments, the surfactants include an octylphenol-based surfactant, such as Igepal®. Additionally, nucleic acids may be amplified or separated under various controlled temperatures and temperature gradients.

The methods of the present disclosure may be utilized to amplify various target nucleic acids. For instance, in some embodiments, the target nucleic acids include, without limitation, DNA, RNA, DNA encoding ribosomal RNA or a ribosomal protein (e.g., 16S), intergenic regions (e.g., ITS 2), genes encoding nucleoproteins, genes encoding capsid proteins, genes encoding toxins, genes encoding antimicrobial resistance, or combinations thereof.

In some embodiments, the sample pre-treatment step includes mixing the nucleic acids with one or more detection dyes. In some embodiments, the one or more detection dyes include ethidium bromide, SYBR safe, or combinations thereof. In some embodiments, the sample pre-treatment step includes mixing nucleic acids with one or more loading dyes. In some embodiments, the one or more loading dyes include xylene cyanol, bromophenol blue, or combinations thercof.

### Separation of Nucleic Acids

The methods of the present disclosure may be utilized to separate various types of nucleic acids from samples. For instance, in some embodiments, the nucleic acids include DNA, double-stranded DNA, single-stranded DNA, RNA, double-stranded RNA, single-stranded RNA, mRNA, tRNA, labeled nucleic acids (e.g., biotinylated nucleic acids), unlabeled nucleic acids, or combinations thereof.

The nucleic acids to be separated may be in various forms. For instance, in some embodiments, the nucleic acids are in non-amplified form. In some embodiments, the nucleic acids are in amplified form. In some embodiments, the nucleic acids are amplified by the methods described herein. For instance, in some embodiments, the nucleic acids are in PCR-amplified form. In some embodiments, the nucleic acids are in competitive PCR-amplified form. In some embodiments, the nucleic acids arc in bound form. For instance, in some embodiments, the nucleic acids are bound to at least an antibody, lectin, affibody, protein, particle, dye, fluor, nanoparticle, virus, bacteriophage, aptamer, or combinations thereof.

The methods of the present disclosure may be utilized to separate nucleic acids within various periods of time. For instance, in some embodiments, the nucleic acids are separated in less than about 2.5 minutes. In some embodiments, the nucleic acids are separated in less than about 8 minutes. In some embodiments, the nucleic acids are separated in less than about 6 minutes.

### Nucleic Acid Detection

In some embodiments, the methods of the present disclosure also include a step of detecting one or more of the separated nucleic acids. In some embodiments, the nucleic acid detection step includes locating one or more of the separated nucleic acids. In some embodiments, the nucleic acid detection step includes identifying one or more of the separated nucleic acids. In some embodiments, the nucleic acid detection step includes manual detection of one or more of the separated nucleic acids through visualization. In some embodiments, the nucleic acid detection step includes quantifying one or more of the separated nucleic acids.

In some embodiments, the nucleic acid detection step includes automated detection of one or more of the separated nucleic acids through the utilization of a nucleic acid detection system. In some embodiments, the nucleic acid detection system includes an optical system. In some embodiments, the optical system includes a camera. In some embodiments, the camera is operable to capture vidcos and/or images from a fiber-based electrophoresis medium.

In some embodiments, the nucleic acid detection system includes a software operational to detect one or more of the separated nucleic acids. For instance, in some embodiments, captured videos and/or images may be analyzed by various image analysis software from a fiber-based electrophoresis medium. In some embodiments, the software includes an image analysis software, a machine-learning algorithm trained on the one or more separated nucleic acids, or combinations thereof. In some embodiments, the software includes a machine-learning algorithm trained on the migration patterns of one or more of the separated nucleic acids. In some embodiments, the software can identify successful or unsuccessful separation or imaging, errors, or faults.

### Diagnosis of a Disease or Condition

In some embodiments, the methods of the present disclosure also include a step of diagnosing a disease or condition based on the detection of one or more of the separated nucleic acids. For instance, in some embodiments, the methods of the present disclosure may be utilized to diagnose a certain disease or condition based on the migration pattern of one or more of the separated nucleic acids across the fiber-based electrophoresis medium. In some embodiments, the methods of the present disclosure may be utilized to diagnose a certain disease or condition at a point of care.

In some embodiments, the disease or condition includes a pathogenic infection. In some embodiments, the pathogenic infection includes, without limitation, a viral infection, a bacterial infection, a yeast infection, a protozoal infection, a fungal infection, or combinations thereof.

In some embodiments, the disease or condition includes genetic condition, such as a chromosomal alteration, a pathogenic genetic condition, or combinations thereof. In some embodiments, the genetic condition includes, without limitation, cancer, sickle cell disease, enzyme deficiency disease, thalassemia, or combinations thereof.

In some embodiments, the disease or condition includes a viral infection. In some embodiments, the viral infection includes, without limitation, a hepatitis B virus (HBV) infection, a hepatitis C virus (HCV) infection, a papilloma virus infection, a human immunodeficiency virus infection (HIV), an influenza virus infection, an ebola virus infection, a respiratory syncytial virus (RSV) infection, a variola virus infection (e.g., a smallpox or other pox-related virus infection), a viral hemorrhagic fever virus infection, an arenavirus infection, a Junin virus infection, a Machupo virus infection, a Guanarito virus infection, a Chapare virus infection, a Lassa virus infection, a Lujo virus infection, a Bunyavirus infection, a Hantavirus infection (e.g., a viral infection causing Hanta Pulmonary syndrome), a Rift Valley Fever virus infection, a Crimean Congo Hemorrhagic Fever virus infection, a Flavivirus infection, a Dengue virus infection, a Filovirus infection, an Ebola infection, a Marburg virus infection, a transplant-associated viral infection, or combinations thereof. In some embodiment, the methods of the present disclosure may be utilized to detect the viral load of the viral infection.

In some embodiments, the disease or condition includes a bacterial, yeast, protozoal or fungal infection. In some embodiments, the infection includes, without limitation, infection by tuberculosis, staphylococcus, streptococcus bacteria, leptospirosis, blastomycosis, candida, Burkholderia, cholera, rickettsia, Coxiella, Bacillus anthracis (anthrax), Yersinia pestis (plague), Francisella tularensis (tularemia), diarrheagenic E. coli, Pathogenic Vibrio, Shigella, Salmonella, Listeria monocytogenes, Campylobacter jejuni, Yersinia enterocolitica, Cryptosporidium parvum, Cyclospora cayatanensis, Giardia lamblia, Entamoeba histolytica, Toxoplasma gondii, or combinations thereof. In some embodiments, the methods of the present disclosure may be utilized to perform a quantitative analysis of a detected disease. For instance, in some embodiments, the methods of the present disclosure may quantify the concentration or level of detected nucleic acids. The quantified results may then be utilized to assist with a treatment decision.

### Treatment Decision

In some embodiments, the methods of the present disclosure also include a step of implementing a treatment decision based on the diagnosis of the disease or condition. For instance, in some embodiments, the treatment decision may include, without limitation, monitoring the disease or condition, administrating a therapeutic agent to the subject, or combinations thereof. In some embodiments, the methods of the present disclosure also include a step of repeating the nucleic acid separation after implementing the treatment decision. In some embodiments, the methods of the present disclosure also include a step of conducting a different analysis after implementing the treatment decision or a first analysis.

### Nucleic Acid Separation Systems

Additional embodiments of the present disclosure pertain to nucleic acid separation systems for separating nucleic acids. In some embodiments, the nucleic acid separation systems of the present disclosure include a fiber-based electrophoresis medium that is operational to receive a sample containing nucleic acids. In some embodiments, the nucleic acid separation systems of the present disclosure also include a first electrode and a second electrode operational to generate an electric field across the fiber-based electrophoresis medium. In some embodiments, the generated electric field induces migration and separation of the nucleic acids across the fiber-based electrophoresis medium.

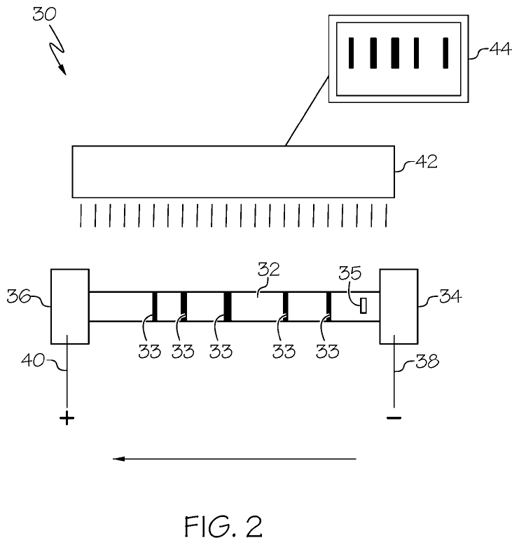

An example of a nucleic acid separation system is illustrated in FIG. 2 as nucleic acid separation system 30. Nucleic acid separation system 30 includes a fiber-based electrophoresis medium 32 that is operational to receive a sample containing nucleic acids through receptacle 35. Nucleic acid separation system 30 also includes a first container 34 positioned on a first end of the fiber-based electrophoresis medium, and a second container 36 positioned on a second end of the fiber-based electrophoresis medium.

Nucleic acid separation system 30 also includes a first electrode 38 (anode) and a second electrode 40 (cathode) that are positioned within containers 34 and 36, respectively. Additionally, nucleic acid separation system 30 includes a nucleic acid detection system 42 that is operational to optically detect and track nucleic acids 33 on fiber-based electrophoresis medium 32. Nucleic acid separation system 30 also includes a display 44 that is operational to display information related to one or more of the separated nucleic acids 33.

In operation, containers 34 and 36 are filled with a buffer. Next, a sample containing nucleic acids is placed in receptable 35. Thereafter, electrodes 38 and 40 are actuated to generate an electric field across the fiber-based electrophoresis medium 32. The generated electric field induces migration and separation of nucleic acids 33 across the fiber-based electrophoresis medium 32. Thereafter, nucleic acid detection system 42 optically detects and tracks nucleic acids 33 on fiber-based electrophoresis medium 32. Subsequently, display 44 displays information related to one or more of the separated nucleic acids 33. As set forth in more detail herein, the nucleic acid separation systems of the present disclosure can have numerous embodiments.

### Containers

In some embodiments, the nucleic acid separation systems of the present disclosure may include one or more containers. In some embodiments, the one or more containers are operational to provide the fiber-based electrophoresis medium with buffer. In some embodiments, the one or more containers include a first container positioned on a first end of the fiber-based electrophoresis medium, and a second container positioned on a second end of the fiber-based electrophoresis medium (e.g., containers 34 and 36 in FIG. 2). In some embodiments, the nucleic acid separation systems of the present disclosure include a single container that is associated with the first and second fibers of a fiber-based electrophoresis medium.

### Fiber-Based Electrophoresis Medium

The nucleic acid separation systems of the present disclosure may include various types of fiber-based electrophoresis media. For instance, in some embodiments, the fiber-based electrophoresis medium includes a paper-based electrophoresis medium. In some embodiments, the fiber-based electrophoresis medium includes, without limitation, cellulose acetate, cellulose, nitrocellulose, polyvinylidene difluoride (PVDF), nylon, or combinations thercof. In some embodiments, the fiber-based electrophoresis medium includes cellulose acetate. In some embodiments, the fiber-based electrophoresis medium is in the form of a microchip.

### Nucleic Acid Detection Systems

In some embodiments, the nucleic acid separation systems of the present disclosure may also include a nucleic acid detection system (e.g., nucleic acid detection system 42 in FIG. 2). In some embodiments, the nucleic acid detection system is operational to optically detect and track nucleic acids on a fiber-based electrophoresis medium.

In some embodiments, the nucleic acid detection system includes an optical system. In some embodiments, the optical system includes a light source. In some embodiments, the light source is operational to emit light on a fiber-based electrophoresis medium for detection of nucleic acids. In some embodiments, the light source includes, without limitation, light emitting diodes (LEDs), flash lamps, or combinations thereof.

In some embodiments, the optical system includes a camera. In some embodiments, the camera is operational to capture one or more images of the separated nucleic acids.

In some embodiments, the nucleic acid detection system includes a software operational to detect one or more of the separated nucleic acids. In some embodiments, the software includes an image analysis software, a machine-learning algorithm trained on the one or more separated nucleic acids, or combinations thereof. In some embodiments, the software includes a machine-learning algorithm trained on the migration patterns of one or more of the separated nucleic acids.

### Display

In some embodiments, the nucleic acid separation systems of the present disclosure may also include a display (e.g., display 44 in FIG. 2). In some embodiments, the display is electrically connected to a nucleic acid detection system. In some embodiments, the display is operational to display information related to one or more of the separated nucleic acids.

### Nucleic Acid Separation Capacities

The nucleic acid separation systems of the present disclosure may include various advantageous nucleic acid separation capacities. For instance, in some embodiments, the nucleic acid separation system is operational to separate nucleic acids in less than about 2.5 minutes. In some embodiments, the nucleic acid separation system is operational to separate nucleic acids in less than about 8 minutes. In some embodiments, the nucleic acid separation system is operational to separate nucleic acids in less than about 6 minutes.

The nucleic acid separation systems of the present disclosure may be powered by various voltage sources. For instance, in some embodiments, the nucleic acid separation systems of the present disclosure may be powered by battery. In some embodiments, the nucleic acid separation systems of the present disclosure may be powered by solar cells. In some embodiments, the nucleic acid separation systems of the present disclosure may be powered by electricity.

### Methods of Making Nucleic Acid Separation Systems

Additional embodiments of the present disclosure pertain to methods of making the nucleic acid separation systems of the present disclosure. In some embodiments, such methods include associating a fiber-based electrophoresis medium of the present disclosure with first and second electrodes. In some embodiments, the fiber-based electrophoresis medium is operational to receive a sample that includes nucleic acids. In some embodiments, the first and second electrodes are operational to generate an electric field across the fiber-based electrophoresis medium. In some embodiments, the generated electric field is operational to induce migration and separation of the nucleic acids across the fiber-based electrophoresis medium.

In some embodiments, the methods of the present disclosure also include a step of associating a fiber-based electrophoresis medium with one or more containers of the present disclosure. In some embodiments, the methods of the present disclosure also include a step of associating the fiber-based electrophoresis medium with a nucleic acid detection system of the present disclosure. In some embodiments, the methods of the present disclosure also include a step of electrically connecting the nucleic acid detection system with a display of the present disclosure.

### Applications and Advantages

The nucleic acid separation systems and methods of the present disclosure can have numerous advantages. For instance, in some embodiments, the systems and methods of the present disclosure allow users to separate nucleic acids based on their size (bp) and charge, thereby allowing for the identification and quantification of nucleic acid molecules of different sizes based on their locations and migration patterns. Moreover, unlike conventional gel-based nucleic acid separation approaches, the systems and methods of the present disclosure enable rapid (e.g., less than 8 minutes), low cost, facile and portable mechanisms to separate nucleic acids.

As such, the methods and systems of the present disclosure can have numerous applications. For instance, in some embodiments, the methods and systems of the present disclosure allow users to separate and detect nucleic acids in a portable manner in various settings, such as at various points of care. Moreover, the methods and systems of the present disclosure can allow users to diagnose various diseases.

### ADDITIONAL EMBODIMENTS

Reference will now be made to more specific embodiments of the present disclosure and experimental results that provide support for such embodiments. However, Applicant notes that the disclosure below is for illustrative purposes only and is not intended to limit the scope of the claimed subject matter in any way.

### Example 1. Separation of DNA through the Utilization of Cellulose Acetate Paper

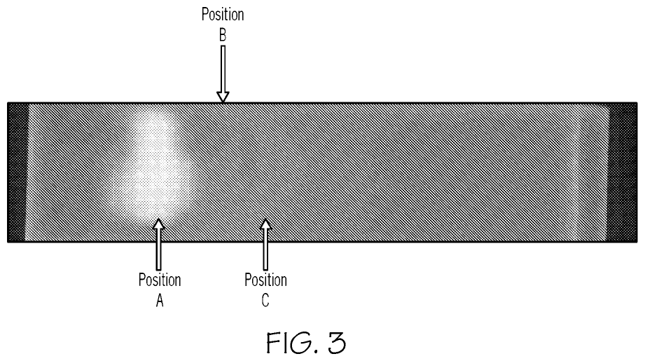

In this Example, Applicant utilized a microchannel containing cellulose acetate paper pre-wetted with a pretreatment buffer to separate DNA. Both ends of the cellulose acetate paper were immersed in buffer ports. The buffer ports received running buffer and contained electrodes for generating electric fields through the electrode-buffer-cellulose acetate. An image of the cellulose acetate paper with the separated DNA is shown in FIG. 3.

DNA of different sizes (i.e., 25 Bp and 100 Bp) were pre-mixed with ethidium bromide. As illustrated in FIG. 3, the mixture was loaded at position A of the cellulose acetate paper. Next, the cellulose acetate paper was loaded into a Gazelle reader to apply electric field. In particular, a voltage of 250 V was applied across the 4 cm-long cellulose acetate paper for 5 minutes. Three (3) bands were observed at the end of the separation. Without being bound by theory, Applicant believes that the band at position A in FIG. 3 includes the ethidium bromide that was not bound to DNAs. The bands at positions B and C in FIG. 3 represent the 100 Bp DNA and 25 Bp DNA, respectively.

### Example 2. Quantitative Nucleotide Detection through Competitive PCR

One application of the paper-based nucleotide electrophoresis technology described in this Application is to conduct quantitative or semi-quantitative nucleotide (i.e., RNA and/or DNA) detection in combination with competitive PCR technology. To help understand this, one potential use is for determining viral load. Viral load (VL) testing is of paramount importance in the management and understanding of various viral infections, including hepatitis B virus, hepatitis C virus, and human immunodeficiency virus (i.e., HBV, HCV, and HIV, respectively), as well as transplant-associated viruses. VL tests provide critical information including discase diagnosis, disease progression monitoring, treatment guidance, assessing viral resistance, management of chronic infections, transplant medicine, and transmission prevention. For example, over half of the 37 million people living with immunodeficiency virus (HIV) are accessing antiretroviral therapy (ART), and the World Health Organization (WHO) recommends monitoring ART effectiveness with routine HIV VL testing.

The current gold standard for measuring VL is quantitative nucleic acid amplification testing using reverse transcriptase polymerase chain reaction (qRT-PCR). However, qRT-PCR tests require state-of-the-art laboratory infrastructure, which are typically scarce or non-existent in low resource settings. Additionally, the outcome of real-time PCR is heavily influenced by the quality of the initial sample template and by the well-known existence of random tube-to-tube variations in the first phases of the amplification process, which occur even under the most finely tuned experimental conditions.

Competitive PCR allows for co-amplification of a target nucleic acid sample alongside an exogenous competitor, which shares a highly similar sequence. The target and competitor templates are subject to the same predictable and unpredictable variables that might affect RT and PCR, and thus amplify at the same rate. Therefore, competitive PCR facilitates precise quantification. While competitive PCR is a powerful tool for absolute nucleic acid quantification, it requires post-PCR electrophoresis-based detection and analysis.

Therefore, one application of the paper-based nucleotide electrophoresis technology in this Example is to use the technology as a detection method to separate and quantify the DNA amplicons that were output from competitive PCR. For instance, in an embodiment, the competitor DNA may be designed to have a distinct size (or charge to mass ratio) from the target HIV DNA amplicon. The overall negative net charges of the tracking dyes, DNA amplicons, and competitor DNA may then cause them to travel toward the positive electrode when placed in an electric field. Differences of electrophoretic mobilities between the markers, HIV DNA amplicons, competitor DNA amplicons, and non-specifically amplified amplicons allow separation within the paper as the sieving medium. The amplification factor achieved by the PCR can then be determined by the relative abundance of the competitor DNA to the electrophoretic marker. Finally, the concentration of HIV DNA can be determined based on its relative abundance compared to the competitor DNA. HIV DNA, competitor DNA, and non-specifically amplified amplicons based on their location can be determined to increase test specificity. Morcover, implementation of competitor DNA may compensate for variations during PCR amplification to increase quantification accuracy.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

1. A method of separating nucleic acids from a sample, said method comprising:
applying the sample to a fiber-based electrophoresis medium; and
generating an electric field across the fiber-based electrophoresis medium, wherein the generated electric field induces migration and separation of the nucleic acids across the fiber-based electrophoresis medium.

2. The method of claim 1, wherein a first electrode and a second electrode are utilized to generate the electric field across the fiber-based electrophoresis medium.

3. The method of claim 1, wherein the fiber-based electrophoresis medium comprises a paper-based electrophoresis medium.

4. The method of claim 1, wherein the fiber-based electrophoresis medium is selected from the group consisting of cellulose acetate, cellulose, nitrocellulose, polyvinylidene difluoride (PVDF), nylon, or combinations thereof.

5. The method of claim 1, wherein both ends of the electrophoresis medium are immersed in containers comprising a buffer.

6. The method of claim 1, wherein the sample comprises a biological sample derived from a subject.

7. The method of claim 1, further comprising a step of pre-treating the sample.

8. The method of claim 7, wherein the sample pre-treatment step comprises purifying the nucleic acids from the sample, amplifying the nucleic acids, labeling the nucleic acids, mixing the nucleic acids with one or more detection dyes, mixing the nucleic acids with loading dyes, or combinations thereof.

9. The method of claim 7, wherein the sample pre-treatment step comprises purifying the nucleic acids from the sample

10. The method of claim 7, wherein the pre-treatment step comprises amplifying the nucleic acids.

11. The method of claim 10, wherein the amplifying step occurs by a method selected from the group consisting of isothermal amplification, loop-mediated isothermal amplification (LAMP), recombinase polymerase amplification (RPA), helicase-dependent amplification (HDA), rolling circle amplification (RCA), whole-genome amplification (WGA), multiple displacement amplification (MDA), nucleic acid sequence based amplification (NASBA), linear amplification, reverse transcription-based amplification, competitive amplification, polymerase chain reaction (PCR), competitive PCR, reverse transcription PCR (RT-PCR), asymmetric PCR, or combinations thereof.

12. The method of claim 10, wherein the amplifying step comprises competitive amplification.

13. The method of claim 12, wherein the competitive amplification process comprises competitive PCR, competitive isothermal amplification, competitive reverse transcription, competitive RPA, or combinations thereof.

14. The method of claim 1, wherein the nucleic acids comprise DNA, double-stranded DNA, single-stranded DNA, double-stranded RNA, single-stranded RNA, mRNA, tRNA, labeled nucleic acids, unlabeled nucleic acids, or combinations thereof.

15. The method of claim 1, further comprising a step of detecting one or more of the separated nucleic acids.

16. The method of claim 15, wherein the nucleic acid detection step comprises quantifying one or more of the separated nucleic acids.

17. The method of claim 15, wherein the nucleic acid detection step comprises automated detection of one or more of the separated nucleic acids through the utilization of a nucleic acid detection system, wherein the nucleic acid detection system comprises: an optical system comprising a light source operational to emit light on the fiber-based electrophoresis medium for detection of nucleic acids, and a camera operational to capture one or more images of the separated nucleic acids; and a software operational to detect one or more of the separated nucleic acids.

18. The method of claim 1, further comprising a step of diagnosing a disease or condition based on the detection of one or more of the separated nucleic acids.

19. The method of claim 18, wherein the diagnosis is based on the migration pattern of one or more of the separated nucleic acids across the fiber-based electrophoresis medium.

20. The method of claim 18, wherein the disease or condition comprises a pathogenic infection, a genetic condition, or combinations thereof.

21. The method of claim 20, wherein the disease or condition comprises a pathogenic infection selected from the group consisting of a viral infection, a bacterial infection, a yeast infection, a protozoal infection, a fungal infection, or combinations thereof.

22. The method of claim 18, further comprising a step of implementing a treatment decision based on the diagnosis of the disease or condition.

23. A nucleic acid separation system for separating nucleic acids, said system comprising:
a fiber-based electrophoresis medium operational to receive a sample comprising nucleic acids; and
a first electrode and a second electrode operational to generate an electric field across the fiber-based electrophoresis medium, wherein the generated electric field induces migration and separation of the nucleic acids across the fiber-based electrophoresis medium.

17. The nucleic acid separation system of claim 23, further comprising a first container positioned on a first end of the fiber-based electrophoresis medium, and a second container positioned on a second end of the fiber-based electrophoresis medium.

25. The nucleic acid separation system of claim 23, wherein the fiber-based electrophoresis medium comprises a paper-based electrophoresis medium.

26. The nucleic acid separation system of claim 23, wherein the fiber-based electrophoresis medium is selected from the group consisting of cellulose acetate, cellulose, nitrocellulose, polyvinylidene difluoride (PVDF), nylon, or combinations thereof.

27. The nucleic acid separation system of claim 23, further comprising a nucleic acid detection system, wherein the nucleic acid detection system is operational to optically detect and track the nucleic acids on the fiber-based electrophoresis medium.

28. The nucleic acid separation system of claim 27, wherein the nucleic acid detection system comprises an optical system comprising a light source operational to emit light on the fiber-based electrophoresis medium for detection of nucleic acids, and a camera operational to capture one or more images of the separated nucleic acids.

29. The nucleic acid separation system of claim 27, wherein the nucleic acid detection system comprises a software operational to detect one or more of the separated nucleic acids.

30. The nucleic acid separation system of claim 27, further comprising a display electrically connected to the nucleic acid detection system, wherein the display is operational to display information related to one or more of the separated nucleic acids.
